(19) United States
(12) Patent Application Publication
(10) Pub. No.: US 2024/0269430 A1
(43) Pub. Date: Aug. 15, 2024

(54) PATTERNED TIE LAYER FOR CATHETER PERFORMANCE OPTIMIZATION

(71) Applicants: STRYKER CORPORATION, Kalamazoo, MI (US); STRYKER EUROPEAN OPERATIONS LIMITED, Carrigtwohill (IE)

(72) Inventors: Clay W. Northrop, Salt Lake City, UT (US); Ted W. Layman, Park City, UT (US)

(73) Assignees: STRYKER CORPORATION, Kalamazoo, MI (US); STRYKER EUROPEAN OPERATIONS LIMITED, Carrigtwohill, Co. Cork (IE)

(21) Appl. No.: 18/647,822

(22) Filed: Apr. 26, 2024

Related U.S. Application Data
(63) Continuation of application No. PCT/US2022/078927, filed on Oct. 28, 2022.
(60) Provisional application No. 63/278,463, filed on Nov. 11, 2021.

(51) Int. Cl. A61M 25/00

(52) U.S. Cl. CPC A61M 25/0054; A61M 25/0013; A61M 25/0045

(57) ABSTRACT

An intravascular catheter comprises a hypotube structure having an elongate tubular body with a proximal end, a distal end, a hypotube pattern of apertures and solid elements disposed on the distal end of the tubular body, and a hypotube lumen extending between the proximal end and the distal end of the tubular body. The intravascular catheter further comprises an inner polymer liner disposed within the hypotube lumen, the inner polymer liner having a liner lumen. The intravascular catheter further comprises a tie layer intermittently attaching the inner polymer liner to the solid elements of the hypotube structure at least one discrete adhesion region along a length of the distal end of the tubular body.

### RELATED APPLICATION DATA

This application is a continuation of International Patent Application No. PCT/US2022/078927, filed Oct. 28, 2022, which claims priority to U.S. Provisional Patent Application No. 63/278,463, filed Nov. 11, 2021, the disclosures of all of which are hereby incorporated herein by reference in their entirety into the present application.

### FIELD

The present disclosure relates generally to medical devices, and, more particularly, to a medical catheter.

### BACKGROUND

The use of intravascular catheters for accessing and treating various types of diseases, such as vascular defects, is well-known. For example, a suitable intravascular catheter may be inserted into the vascular system of a patient. A commonly used vascular application to access a target site in a patient involves inserting a guidewire through an incision in the femoral artery near the groin, and advancing the guidewire until it reaches the target site. Then, a catheter is advanced over the guidewire via a lumen in the catheter until an open distal end of the catheter is disposed at the target site. Simultaneously or after placement of the distal end of the catheter at the target site, an intravascular implant is advanced through the lumen of the catheter via a delivery wire.

In certain applications, such as neurovascular treatment, the catheters are required to navigate tortuous and intricate vasculature. By using an appropriately sized device having the requisite performance characteristics, such as “pushability,” “steerability,” and “torqueability,” and most important, distal tip flexibility, virtually any target site in the vascular system may be accessed, including that within the tortuous cerebral and peripheral vasculature. The forces applied at the proximal end of these catheters should be transferred to the distal ends for suitably pushability (axial rigidity) and torqueability (rotation). Achieving a balance between these features is highly desirable, but difficult.

For such applications of neurovascular treatment, as well as other applications that involve passing various other devices, agents, and/or fluids into a body lumen or cavity in a patient by the catheter, the properties of the inner surface of the lumen(s) of the catheter may significantly impact the performance of the catheter. In particular, the lubricity of the inner surface may affect the ability to pass other devices, agents, and/or fluids through the lumen(s) of the catheter.

To enhance lubricity, a low friction liner (e.g., polytetrafluoroethylene (PTFE), expanded PTFE (ePTFE, e.g., unidirectional ePTFE or bi-directional ePTFE), a fluoropolymer, perfluoroalkyoxy, alkane (PFA), fluorinated ethylene polyethylene (FEP), polyethylene (PE), or any combination thereof) can surround the lumen of a catheter. The liner may provide a lubricious inner surface to facilitate passing guidewires, pacing leads, or other devices through the lumen of the catheter. Constructing such a catheter, however, is complicated due to the difficulty of bonding the low friction liner to the outer jacket of the catheter. For example, PTFE, in its native form is nearly impossible to bond. Improperly integrating a liner into a catheter may result in delamination, a challenging failure mode in catheter construction that carries both risk and cost burden for many device manufacturers. Detection typically occurs during final testing, after production of the complete catheter assembly, resulting in significant final product yield loss. More importantly, delamination can lead to failures in the field and product recalls.

To prevent delamination between the inner liner and the outer jacket of a catheter, a tie layer in the form of an ultrathin thermoplastic coating may be applied over an inner polymer liner during catheter construction. This tie layer creates a melt-bondable substrate that improves adhesion to both the inner polymer liner and the outer jacket of the catheter. Alternately, another form of adhesive could be used, such as a liquid, dispersion, or solid.

Presently, numerous microcatheter designs exist that possess hypotubes utilized within the construction. In general, a hypotube is a long thin-walled tube formed from a metal or a metal alloy, such stainless steel, nickel titanium alloy (e.g., nitinol), rigid plastics, or the like. A hypotube often has micro-engineered features along its length. The distal ends of hypotubes may have a slotted pattern that enhances their flexibility, while providing sufficient axial rigidity to maintain the pushability of the hypotubes through the vasculature of a patient. As discussed above, it is desirable to incorporate some type of inner liner with a slotted hypotube to provide a low friction interface with devices that are pushed through the hypotube. Such a liner may be slightly undersized so that it slides inside the slotted hypotube during manufacturing. In other embodiments, the slotted tube may have a reinforcement that may give the liner more support and integrity as the catheter navigates the vasculature to a treatment location. In some instances, a polymer jacket may be applied to the outer diameter of the slotted hypotube to provide a seal and to also minimize any exterior surface roughness imparted by the slots of the hypotube while still providing flexibility. This outer jacket may fill the aperture/slots in the hypotube, and even coat the internal surface of the hypotube.

However, it is known that even a minimum-wall-thickness PTFE liner can add unacceptable stiffness to the distal end of a slotted hypotube catheter. In particular, in a slotted hypotube structure 1 comprising a pattern of apertures (e.g., slots) 2 and solid elements (e.g., struts) 4 illustrated in FIG. 1, flexibility requires that apertures 2 in the hypotube structure 1 be free to open or close in response to a bending force. If an inner polymer liner 4 is intimately and continuously coupled with the inner surface of the solid elements 3 of the hypotube structure 1 via a tie layer 5, then the inner polymer liner 4 must stretch to allow the apertures 2 to open. This can require a relatively high percentage of elongation, as only the polymer spanning an aperture 2 is available to stretch. In distal regions of the slotted hypotube catheter, the inner polymer liner is by far the dominant element in terms of stiffness.

Not only does the intimate/continuous coupling between an inner liner and the hypotube structure detrimentally affect the flexibility of the distal region of the slotted hypotube catheter, past experience has shown that it has a detrimental effect on the torque transmission and navigability of the catheter structure. In particular, due to the relatively high strain required to bend the distal region of the slotted hypotube catheter, the inner polymer liner may plastically deform, thereby creating a permanent bend in the distal region of the catheter. Torquing of the deformed catheter may cause the distal end of the catheter to whip, thereby detrimentally affecting the steerability of the catheter.

One technique that addresses this issue is to have a “floating” inner polymer liner by bonding discrete locations of the inner polymer liner to the slotted hypotube structure to prevent the liner from bunching up or moving independently of the slotted hypotube structure. However, such a floating liner must be composed of a composite that is reinforced with a metal coil or braid to prevent collapse under vacuum and loosely toleranced (so that the liner may be slid into the hypotube structure) to ensure proper fit within the hypotube structure, both of which increase wall thickness. Furthermore, because the inner polymer liner “floats” within the slotted hypotube structure (i.e., there is space between the inner polymer liner and the slotted hypotube structure between the bonded discrete locations), the outer diameter of the catheter must be increased to maintain patency of the working lumen in the catheter. This floating liner must then be tediously bonded at discrete locations to the hypotube structure through the slots of the hypotube structure, thereby increasing manufacture time.

There, thus, is an ongoing need for an efficient technique that bonds an inner polymer lining to a slotted hypotube structure without unduly increasing the bending stiffness of the slotted hypotube structure.

### BRIEF DESCRIPTION OF THE DRAWINGS

The drawings illustrate the design and utility of preferred embodiments of the disclosed inventions, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. Further, an aspect or an advantage described in conjunction with a particular embodiment of the disclosed inventions is not necessarily limited to that embodiment and can be practiced in any other embodiments even if not so illustrated.

In order to better appreciate how the above-recited and other advantages and objects of the disclosed inventions are obtained, a more particular description of the disclosed inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a longitudinal-sectional view of a prior art slotted hypotube structure;

FIG. 2 is a profile view of one embodiment of an intravascular catheter, particularly showing a distal end of the intravascular catheter in a straight geometry;

FIG. 3 is a profile view of the intravascular catheter of FIG. 2, particularly showing the distal end of the intravascular catheter in a curved geometry

FIG. 4 is a profile view of the distal end of one embodiment of a hypotube structure used in the intravascular catheter of FIG. 2;

FIG. 5 is a perspective view of the distal end of the hypotube structure of FIG. 4;

FIG. 6 is a cross-sectional view of the distal end of one embodiment of a catheter body of the intravascular catheter of FIG. 2;

FIG. 7 is a longitudinal-sectional view of the distal end of the catheter body of FIG. 6, particularly showing one embodiment of a tie layer;

FIG. 8 is a perspective view of the distal end of another embodiment of a hypotube structure used in the catheter body of FIG. 6;

FIG. 9 is a perspective view of one embodiment of an inner polymer liner used in the catheter body of FIG. 6;

FIG. 10 is a longitudinal-sectional view of the distal end of the catheter body of FIG. 6, particularly showing another embodiment of a tie layer;

FIG. 11 is a longitudinal-sectional view of the distal end of the catheter body of FIG. 6, particularly showing still another embodiment of a tie layer;

FIG. 12 is a profile view of the inner polymer liner of FIG. 9, particularly showing one embodiment of discrete adhesion regions;

FIG. 13 is a profile view of the inner polymer liner of FIG. 9, particularly showing another embodiment of discrete adhesion regions;

FIG. 14 is a longitudinal-sectional view of the distal end of the catheter body of FIG. 6, particularly showing yet another embodiment of a tie layer;

FIG. 15 is a cross-sectional view of the distal end of another embodiment of a catheter body of the catheter body of FIG. 6;

FIGS. 16A-16C are longitudinal-sectional views of the distal end of the catheter body of FIG. 15, particularly showing several embodiments of a tie layer;

FIG. 17 is a flow diagram illustrating one method of manufacturing the intravascular catheter of FIG. 2;

FIG. 18 is a perspective view of one tubular body used to make the hypotube structure in accordance with the flow diagram of FIG. 17;

FIG. 19 is a perspective view of a pattern of apertures and solid elements formed on the distal end of the tubular body of FIG. 18 to create a hypotube structure;

FIG. 20 is a perspective view of one polymer tube;

FIG. 21 is a perspective view of the polymer tube of FIG. 20 disposed in the inner lumen of the hypotube structure of FIG. 19 in accordance with the flow diagram of FIG. 17;

FIG. 22 is a perspective view of the polymer tube intermittently attached to the hypotube structure via a plurality of discrete adhesion regions in accordance with the flow diagram of FIG. 17;

FIG. 23 is a perspective view illustrating one technique for applying a tie layer to the polymer tube of FIG. 20;

FIG. 24 is a perspective view illustrating another technique for applying a continuous tie layer to the polymer tube of FIG. 20;

FIG. 25 is a perspective view illustrating a technique a technique for applying an adhesive pattern onto the continuous tie layer of FIG. 24;

FIG. 26 is a perspective view illustrating a technique for applying a pattern of adhesive material onto the continuous tie layer of FIG. 24;

FIG. 27 is a perspective view illustrating another technique for applying a pattern of adhesive material onto the continuous tie layer of FIG. 24; and

FIG. 28 is a longitudinal-sectional view of the distal end of the intravascular catheter of FIG. 2, particularly showing another technique for applying a tie layer to the polymer tube of FIG. 20.

### DETAILED DESCRIPTION OF THE ILLUSTRATED EMBODIMENTS

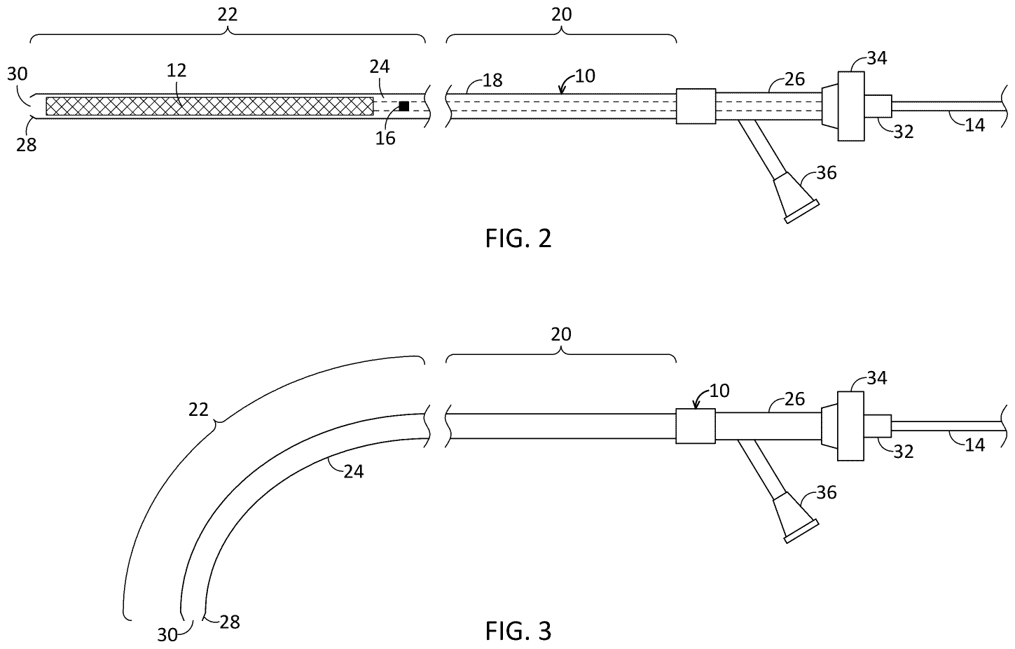

Referring to FIGS. 2 and 3, one embodiment of an intravascular catheter 10 will now be described. The intravascular catheter 10 has a tubular configuration, and can, e.g., take the form of a micro-catheter, a sheath, or the like. In the illustrated embodiment, the intravascular catheter 10 serves as a delivery catheter for delivering a vaso-occlusive device 12 into an aneurysm, although alternative embodiments of the intravascular catheter 10 may deliver other medical devices, e.g., another catheter, a guide member, a stent, a thrombectomy device, etc. Furthermore, other alternative embodiments of the intravascular catheter 10 may serve as a working catheter, e.g., a treatment catheter or diagnostic catheter. A pusher member 14 is detachably coupled to the vaso-occlusive device 12 via a junction 16 (e.g., mechanical, thermal, and hydraulic mechanisms). Thus, the pusher member 14 can be distally advanced to deploy the vaso-occlusive device 12 from the intravascular catheter 10 into an aneurysm (not shown) and selectively detached from the pusher member 14 via action of the junction 16 to deliver the vaso-occlusive device 12 within the aneurysm.

The intravascular catheter 10 generally comprises an elongated catheter body 18 topologically divided between a proximal catheter body section 20 and a distal catheter body section 22, an inner catheter lumen 24 extending within the catheter body 18 from the proximal catheter body section 20 to the distal catheter body section 22, and a proximal catheter hub 26 affixed to the proximal catheter body section 20.

The proximal catheter body section 20 remains outside of the patient and accessible to the operator, while the distal catheter body section 22 is sized and dimensioned to reach remote locations of the vasculature of the patient, and is configured to deliver the vaso-occlusive device 12 to the aneurysm (not shown). The distal catheter body section 22 is more flexible than the proximal catheter body section 20, so that it can transition between a straight configuration (FIG. 2) and a curved configuration (FIG. 3). Generally, the proximal catheter body section 20 may be formed from material that is stiffer than the distal catheter body section 22, so that the proximal catheter body section 20 has sufficient pushability to advance through the patient's vascular system, while the distal catheter body section 22 may be formed of a more flexible material so that the distal catheter body section 22 may remain flexible and track more easily over a guidewire to access remote locations in tortuous regions of the vasculature. In some instances, the proximal catheter body section 20 may include a reinforcement layer, such a braided layer or coiled layer to enhance the pushability of the catheter body 18. The catheter body 18 may optionally comprise an intermediate catheter body section (not shown) that may gradually transition the relatively high bending stiffness of the proximal catheter body section 20 to the relatively low bending stiffness of the distal catheter body section 22. A distal tip 28 of the distal catheter body section 22 may be rounded to minimize the chance of traumatic piercing of body tissue. The intravascular catheter 12 comprises a distal port 30 at the distal tip 28 in communication with the inner catheter lumen 24 and from which the vaso-occlusive device 16 is deployed.

The catheter body 18 has a suitable length for accessing a target tissue site within the patient from a vascular access point. The target tissue site depends on the medical procedure for which the intravascular catheter 10 is used. For example, if the intravascular catheter 10 is used to access vasculature in a brain of a patient from a femoral artery access point at the groin of the patient, the overall length of the catheter body 18 may be 125 cm-200 cm. In one embodiment, the outer diameter of the catheter body 18 may be uniform along the length of the catheter body 18. In another embodiment, the outer diameter of the catheter body 18 may taper in either a gradual fashion or a step-wise fashion from a first outer diameter of the proximal catheter body section 20 to a second outer diameter at the distal catheter body section 22.

The outer diameter of the catheter body 18 may be in the range of 3F-10F. The distal catheter body section 22 may have an outer diameter less than the outer diameter of the proximal catheter body section 20 to reduce the profile of the distal catheter body section 22 and facilitate navigation in tortuous vasculature. Although depicted as having a generally round cross-sectional shape, it can be appreciated that the intravascular catheter 10 can include other cross-sectional shapes or combinations of shapes, e.g., oval, rectangular, triangular, polygonal, and the like. The catheter body 18 is structurally configured for being relatively flexible, pushable, and relatively kink- and buckle-resistant, so that it may resist buckling when a pushing force is applied to the proximal catheter body section 20 to advance the catheter body 18 distally through the vasculature of the patient, and so that it may resist kinking when traversing around a tight turn in the vasculature. The catheter body 18 may be relatively thin-walled, such that it defines a relatively large inner diameter for a given outer diameter, which may further contribute to the flexibility and kink-resistance of the catheter body 18.

In some embodiments, at least a portion of the outer surface of the catheter body 18 includes one or more coatings, such as, e.g., an anti-thrombogenic coating, which may help reduce the formation of thrombi in vitro, an anti-microbial coating, or a lubricating coating (e.g., a hydrophilic coating), which may reduce static friction or kinetic friction between the catheter body 18 and tissue of the patient as the catheter body 18 is advanced through the vasculature or through another catheter.

The diameter of the inner catheter lumen 24 may vary based on the medical procedure for which the intravascular catheter 10 is used, and in the illustrated embodiment, is sized to accommodate the vaso-occlusive device 16. The diameter of the inner catheter lumen 24 may be substantially constant from the proximal catheter body section 20 to the distal catheter body section 22 or may taper from a first diameter at the proximal catheter body section 20 to a second different diameter at the distal catheter body section 22.

The proximal catheter hub 26 may be affixed to the proximal catheter body section 20 using suitable means, e.g., adhesive, welding, etc. The proximal catheter hub 26 comprises a proximal port 32 through which the inner catheter lumen 24 may be accessed, and in some embodiments, closed. For example, the proximal port 32 may be located at a proximal end of the proximal catheter hub 26 and aligned with the inner catheter lumen 24, such that the inner catheter lumen 24 may be accessed via the proximal port 34. In this case, the vaso-occlusive device 12 with the pusher member 14 may be introduced into the inner catheter lumen 24 via the proximal port 34 of the catheter hub 26. The proximal catheter hub 26 may further comprises a side port 36 in fluid communication with the inner catheter lumen 24, which is used to introduce fluids into the catheter body 18. In some embodiments, another structure (not shown) in addition to, or instead of, the proximal catheter hub 26 may be affixed to the proximal catheter body section 20.

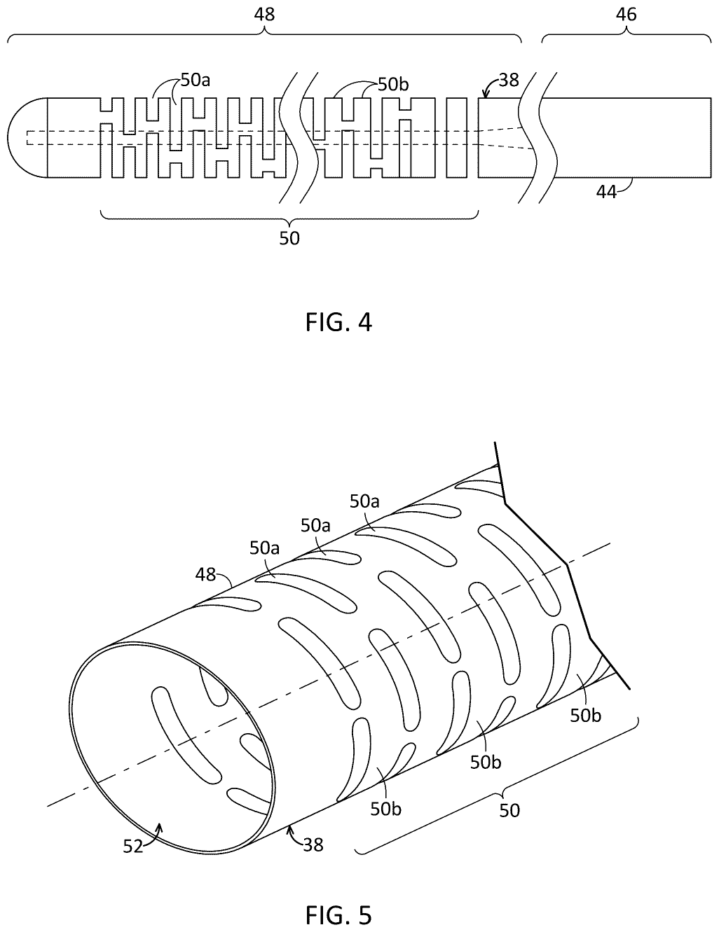

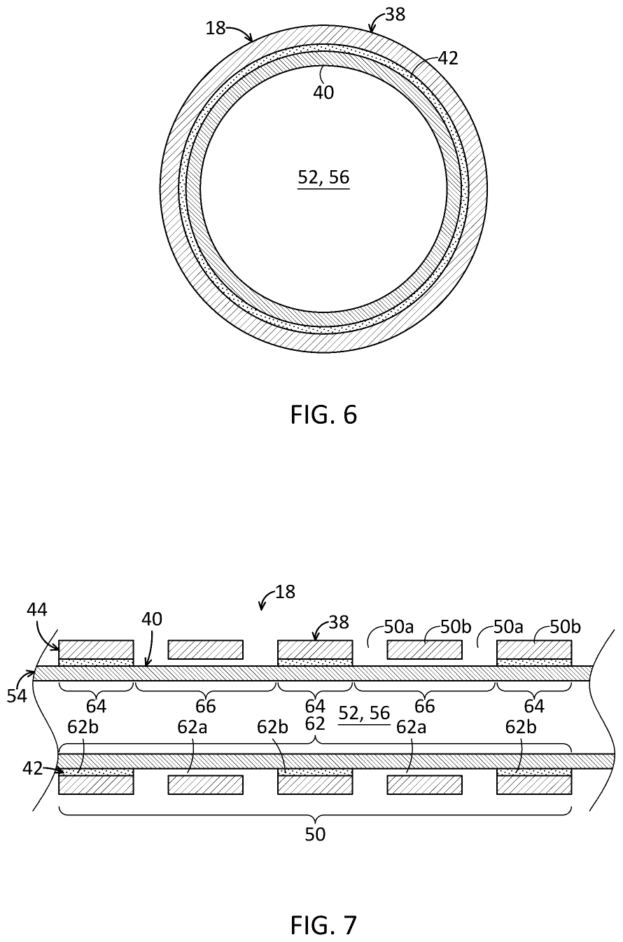

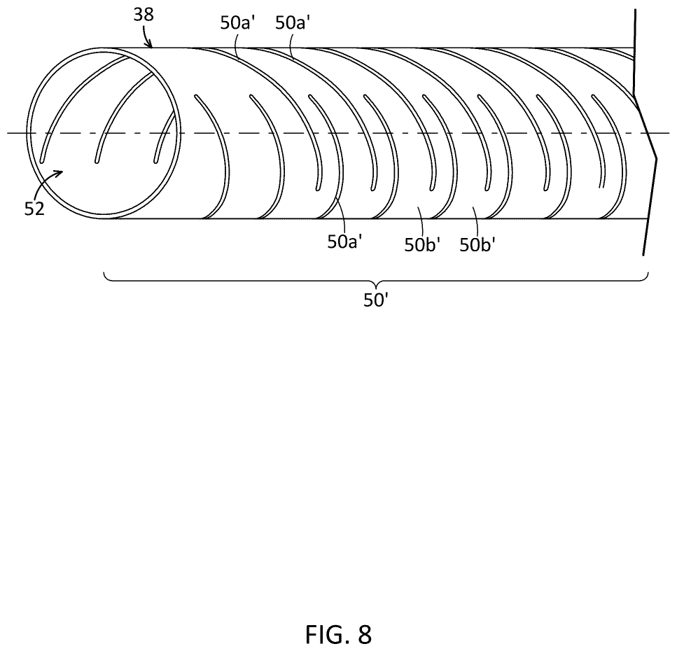

Referring now to FIGS. 4-7, the catheter body 18 of the intravascular catheter 10 generally comprises a hypotube structure 38, an inner polymer liner 40 (shown in FIG. 6-7) disposed within the hypotube structure 38, and a tie layer 42 (shown in FIGS. 6-7) that attaches the inner polymer liner 40 to the hypotube structure 38. Significantly, the bending stiffness of the distal catheter body section 22 (shown in FIG. 3) is reduced through selective application (or removal) of material that forms the tie layer 42, so that a certain distal length of the inner polymer liner 40 is not continuously attached to the hypotube structure 12. In this manner, the navigability of the catheter body 18 through the vasculature of the patient may be improved.

The hypotube structure 38 comprises an elongate tubular body 44 with a proximal end 46 and a distal end 48, a hypotube pattern 50 of apertures 50a and solid elements 50b formed on the distal end 48 of the tubular body 44, and an inner hypotube lumen 52 extending between the proximal end 46 and the distal end 48 of the tubular body 44. The tubular body 44 may be composed of any of a variety of suitable materials, e.g., a material that is rigid, but has some flexibility when used to form extremely thin structures, such as the wall of the tubular body 44. Examples of such materials include metals (e.g., stainless steel, such as 304 stainless steel, 316 stainless steel, 316L stainless steel, nickel chromium (NiCr) steel, nickel titanium alloy (e.g., nitinol), cobalt/chromium), or various plastics. The dimensions of the tubular body 44 may be suitable for one or more desired uses of the intravascular catheter 10. As examples, the outer diameter of the tubular body 44 may be in the range of 0.005-0.080 inches. The inner diameter of the tubular body 44 (i.e., the diameter of the inner hypotube lumen 52) may be in the range of 0.002-0.070 inches.

In the illustrated embodiment, the hypotube pattern 50 takes the form of a brick pattern, and the apertures 50a take the form of slots, and the solid elements 50b take the form of struts. In an alternative embodiment illustrated in FIG. 8, a hypotube pattern 50′ formed on the distal end 48 of the tubular body 44 may have apertures in form of slits 50a′ (which may be oriented circumferentially (perpendicular to the longitudinal axis of the tubular body 44) or helically (at an oblique angle to the longitudinal axis of the tubular body 44), and solid elements in the form of spines 50b′ formed between the slits 50a′.

The hypotube pattern 50 or hypotube pattern 50′ may formed in the distal end 48 of the tubular body 44 by laser cutting, saw cutting (e.g., diamond grit embedded semiconductor dicing blade), etching, waterjet cutting, or electrical discharge machining, among other methods.

In the illustrated embodiment, the hypotube pattern 50 of apertures 50a and solid elements 50b (or hypotube pattern 50′ of apertures 50a′ and solid elements 50b′) are arranged to enhance the bending flexibility of the distal end of the intravascular catheter 10, while maintaining the axial rigidity (pushability) and torqueability of the intravascular catheter 10, such that the intravascular catheter 10 may be introduced and advanced through the tortuous vasculature of a patient. By controlling and varying the spacing, width, and shape of apertures 50a, the bending flexure profile and torsional stiffness of the hypotube structure 38, and thus the distal catheter body section 22 (see FIGS. 2 and 3) may be selectively modified.

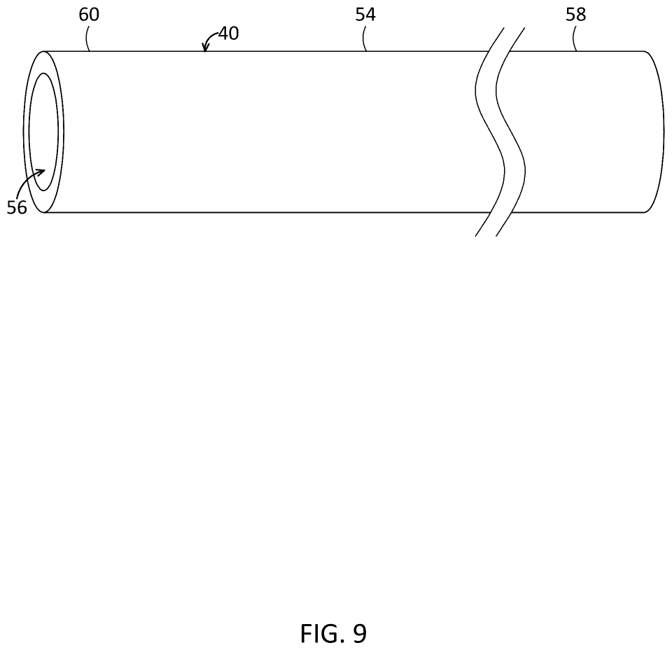

As shown in FIGS. 6-7, the inner polymer liner 40 is disposed within the inner hypotube lumen 52. As further shown in FIG. 9, the inner polymer liner 40 comprises an elongate polymer tube 54 with a proximal end 58 and a distal end 60, and an inner liner lumen 56 extending between the proximal end 58 and the distal end 60 of the polymer tube 54. The inner surface of the polymer tube 54 may be lubricious to facilitate the passage of a medical device (e.g., another catheter, a guide member, an embolic protection device, a stent, a thrombectomy device, or any combination thereof) through the inner liner lumen 56. For example, the material from which the entire polymer tube 54 is formed may be lubricious. Examples of such materials, may include, but are not limited to, polytetrafluoroethylene (PTFE), expanded PTFE (ePTFE, e.g., unidirectional ePTFE or bi-directional ePTFE), a fluoropolymer, perfluoroalkyoxy, alkane (PFA), fluorinated ethylene polyethylene (FEP), polyethylene (PE), or any combination thereof. Other examples of materials from which the polymer tube 54 may be formed included, but are not limited to, Low Density Polyethylene (LDPE) (e.g., about 42D), High Density Polyethylene (HDPE), or any combination thereof.

The polymer tube 54 may have a unitary body construction, i.e., formed as one body, such that the polymer tube 54 is continuous along the entire length of the polymer tube 54. In the preferred embodiment, the polymer tube 54 is unreinforced (meaning that there are no metallic elements disposed within the wall of the polymer tube 54 that function to increase the radial strength of the polymer tube 54), thereby minimizing any bending stiffness imparted by the inner polymer liner 40 onto the distal catheter body section 22 (see FIG. 3). As will be described in further detail below, because the polymer tube 54 is unreinforced, it may be radially expanded within the inner hypotube lumen 52 of the hypotube structure 38, thereby reducing the wall thickness of the polymer tube 54, and ensuring that there is continuous intimate contact (but not continuous coupling) between the polymer tube 54 and the inner hypotube lumen 52 of the hypotube structure 38 (i.e., the hypotube structure 38 is in contact with the entirety of all solid elements of the hypotube structure 38).

The wall thickness of the distal end 60 of the polymer tube 54 may be equal to or less than 001″. In some embodiments, the wall thickness of the polymer tube 54 is substantially constant along a length of the polymer tube 54. In other embodiments, the wall thickness of the polymer tube 54 may decrease toward the distal end 50 (e.g., the thickness of the polymer tube 54 may decrease from the proximal end 48 to the distal end 50 of the polymer tube 54). The inner diameter of the polymer tube 54 (i.e., the diameter of the inner liner lumen 56) may be substantially constant along the entire length of the polymer tube 54. In other embodiments, the inner diameter of the polymer tube 54 may be vary, e.g., may taper continuously from the proximal end 48 to the distal end 50 of the polymer tube 54 or in may vary in a step-wise fashion. In an optional embodiment, the inner polymer liner 40 may have a pattern of apertures (e.g., slots or slits) and solid elements (e.g., struts or ribs) (not shown) to enhance the bending flexibility of the distal catheter body section 22, e.g., as described in U.S. Patent Publication No. 2020/0129733, which is expressly incorporated herein by reference.

The tie layer 42 may be composed of a suitable material, e.g., polyurethane (e.g., Tecoflex™), Pebax®, and nylon. The tie layer 42 may have of a thickness of no more than about 0.005 inches, and in some implementations, approximately 0.001 inches, and perhaps even less than 0.0001 inches. The tie layer 42 may generally extend along at least along a range of 10-20 cm of the distal catheter body section 22, and generally less than about 50 cm along the length of the catheter body 18.

Referring specifically to FIG. 7, the tie layer 42 has a tie layer pattern 62 of apertures 62a and solid elements 62b that is complementary to the hypotube pattern 50, such that the tie layer 42 intermittently attaches the inner polymer liner 40 to the solid elements 50b of the hypotube structure 38 along a length of the hypotube pattern 50. As a result, at least one discrete adhesion region 64 is formed between the hypotube structure 38 and the inner polymer liner 40, and at least one non-adhesion region 66 is formed between the hypotube structure 38 and the inner polymer liner 40. In the illustrated embodiment, the tie layer pattern 62 is complementary to the hypotube pattern 50, such that several discrete adhesion regions 64 are formed between the hypotube structure 38 and the inner polymer liner 40, and several non-adhesion regions 66 are formed between the hypotube structure 38 and the inner polymer liner 40.

Preferably, the tie layer 42 intermittently attaches the inner polymer liner 40 to the solid elements 50b of the hypotube structure 38 along the entire length of the hypotube pattern 50, although in alternative embodiments, the tie layer 42 may intermittently attach the inner polymer liner 40 to the solid elements 50b of the hypotube structure 38 along less than entire length of the hypotube pattern 50, as long as the unreinforced inner polymer liner 40 does not collapse (i.e., belly into the inner hypotube lumen 52) under vacuum. For example, the discrete adhesion region(s) 64 should extend along at least 50% percent, and preferably at least 75%, of the length of the hypotube pattern 50. By intermittently attaching the inner polymer liner 40 to the solid elements 50b of the hypotube structure 38 along a length of the distal end 48 of the tubular body 44, a greater length of the inner polymer liner 40, comprising some percentage of the inner polymer liner 40 spanning the apertures 50a and some percentage of the inner polymer liner 40 underneath the solid elements 50b, is not attached to the hypotube structure 38.

Thus, the total area of the discrete adhesion regions 64 is equal to or less than a certain percentage of the total area of the inner surface of the solid elements 50b of the hypotube structure 38 along a length of the distal end 48 of the tubular body 44 of the hypotube structure 38. In one embodiment, the total area of the discrete adhesion regions 64 is equal to or less than 75% of the total area of the inner surface of the solid elements 50b of the hypotube structure 38 along a length of the distal end 48 of the tubular body 44 of the hypotube structure 38. In another embodiment, the total area of the discrete adhesion regions 64 is equal to or less than 25% of the total area of the inner surface of the solid elements 50b of the hypotube structure 38 along a length of the distal end 48 of the tubular body 44 of the hypotube structure 38.

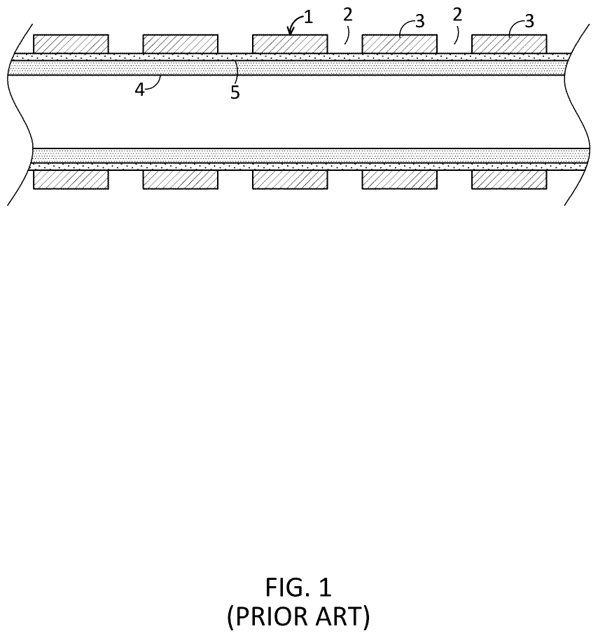

In the embodiment illustrated in FIG. 7, the total area of the discrete adhesion regions 64 is equal to 50% of the total area of the inner surface of the solid elements 50b of the hypotube structure 38 along a length of the distal end 48 of the tubular body 44 of the hypotube structure 38. This should be contrasted with the embodiment of FIG. 1, wherein the inner polymer liner 4 is intimately and continuously coupled with the inner surface of the hypotube structure 3 (i.e., the total area of the adhesion region to which the inner polymer liner 4 is attached to the solid elements 5 of the hypotube structure 3 is 100%). Assuming that the hypotube structure 38 of the embodiment of FIG. 7 is identical to the hypotube structure 3, and the thickness and material from which the inner polymer liner 40 is composed is identical to the thickness and material from which the inner polymer liner 4 is composed, as a result of halving of the total area of the adhesion region between the hypotube structure 38 and the inner polymer liner 40 in the embodiment of FIG. 7, as compared to the total area of the adhesion region between the hypotube structure 3 and the inner polymer liner 4 in the embodiment of FIG. 1, the bending stiffness of the composite structure formed by a hypotube structure and an inner polymer liner could be reduced by nearly a factor of two.

Although FIG. 7 illustrates the discrete adhesion regions 64 as perfectly corresponding to the solid elements 50b of the hypotube structure 38 over the entire length of the distal end 48 of the tubular body 44 of the hypotube structure 38, it should be appreciated that such an arrangement is not required. In one embodiment, the discrete adhesion regions 64 perfectly correspond to the solid elements 50b of the hypotube structure 38 over a very localized length of the distal end 48 of the tubular body 44 of the hypotube structure 38, e.g., 1-2 centimeters.

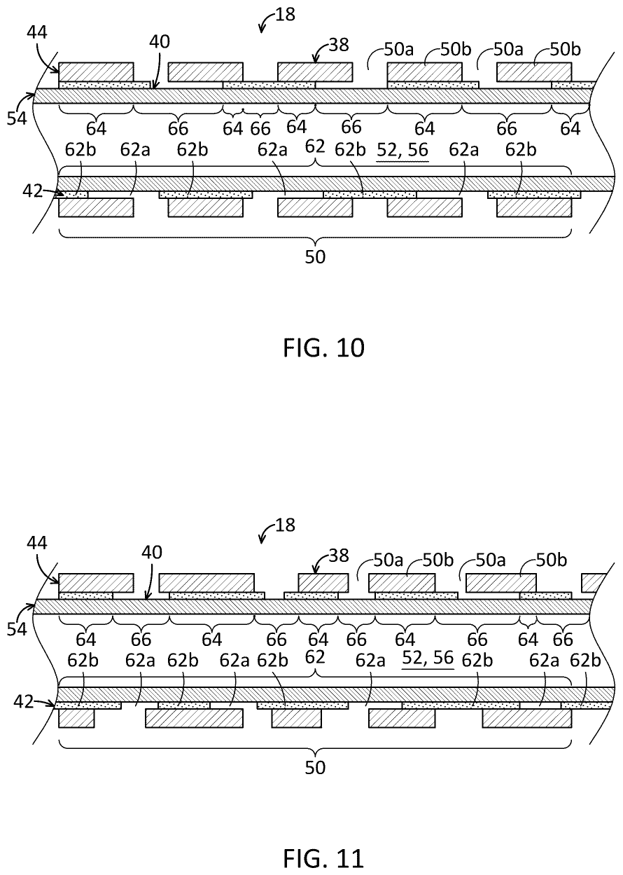

In another embodiment, the hypotube pattern 50 and the tie layer pattern 62 may complement each other, such that total area of discrete adhesion regions 64 formed between the hypotube structure 38 and the inner polymer liner 40 is, on average, equal to or less than a sufficient percentage of the total area of the inner surface of the solid elements 50b of the hypotube structure 38 along a length of the distal end 48 of the tubular body 44 of the hypotube structure 38, as illustrated in FIG. 10. In this case, the total area of the discrete adhesion regions 64 is less than 50% of the total area of the inner surface of the solid elements 50b of the hypotube structure 38 along the hypotube pattern 50. Each of the hypotube pattern 50 and tie layer pattern 62 may be periodic in nature. Because the hypotube pattern 50 and tie layer pattern 62 are predictable, a consistent percentage of total area of the discrete adhesion regions 64 relative to the total area of the inner surface of the solid elements 50b of the solid elements 50b of the hypotube structure 38 may be achieved during manufacture. In this case, the period of the hypotube pattern 50 and the period of the tie layer pattern 62 may be the same as each other or different from each other. In another embodiment, one or both of the hypotube pattern 50 and tie layer pattern 62 may be randomized, as illustrated in FIG. 11.

A pattern of discrete adhesion regions 64 may be formed between the hypotube structure 38 and the inner polymer liner 40 at the intersection of the solid elements 50b of the hypotube pattern 50 and the solid elements 54b of the tie layer pattern 62. The pattern of discrete adhesion regions 64 imparted on the attachment between the inner polymer liner 40 and the hypotube structure 38 could take virtually any form.

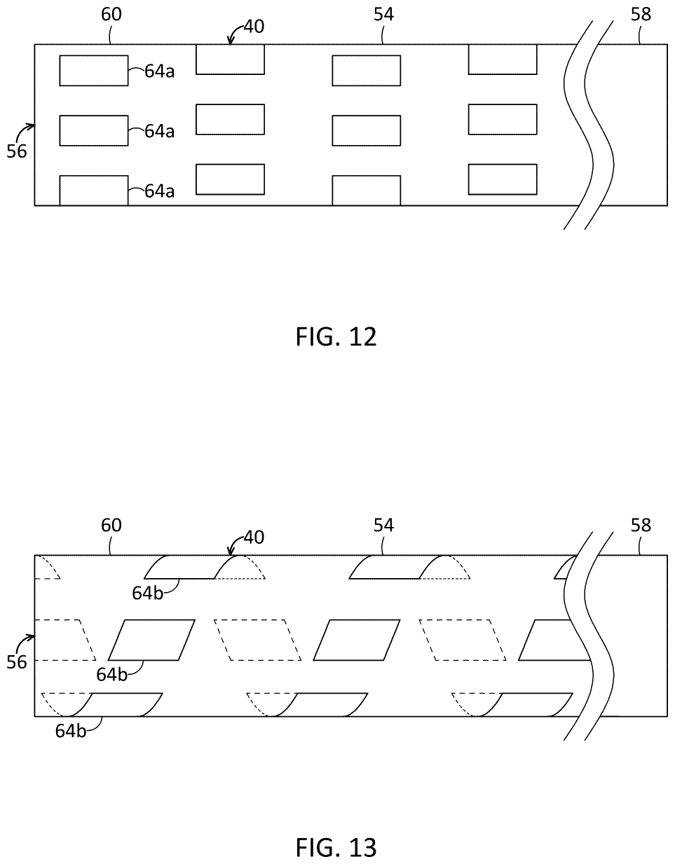

For example, as illustrated in FIG. 12, a circumferential band pattern of discrete adhesion regions 64a may be formed between the hypotube structure 38 (not shown in FIG. 12 for purposes of clarity) and the inner polymer liner 40. In the illustrated embodiment, the circumferential bands of the discrete adhesion regions 64a are straight, although in alternative embodiments, the circumferential bands of discrete adhesion regions 64a may be, e.g., sinusoidal. To form the circumferential band pattern of discrete adhesion regions 64a, the solid elements 62b of the tie layer pattern 62 may be shaped as circumferential bands that periodically intersect the solid elements 50b of the hypotube pattern 50 to form the circumferential band pattern of discrete adhesion regions 64a. As another embodiment, as illustrated in FIG. 13, a spiral pattern of discrete adhesion regions 64b may be formed between the hypotube structure 38 (not shown in FIG. 13 for purposes of clarity) and the inner polymer liner 40. In this case, a solid element 62b of the tie layer pattern 62 may be shaped as a spiral that periodically intersects the solid elements 50b of the hypotube pattern 50 to form the spiral pattern of discrete adhesion regions 64b. Although the spiral pattern of discrete adhesion regions 64b are shown in FIG. 13 as having a constant pitch and width, the spiral pattern of discrete adhesion regions 64b may alternatively have a varying pitch and/or width.

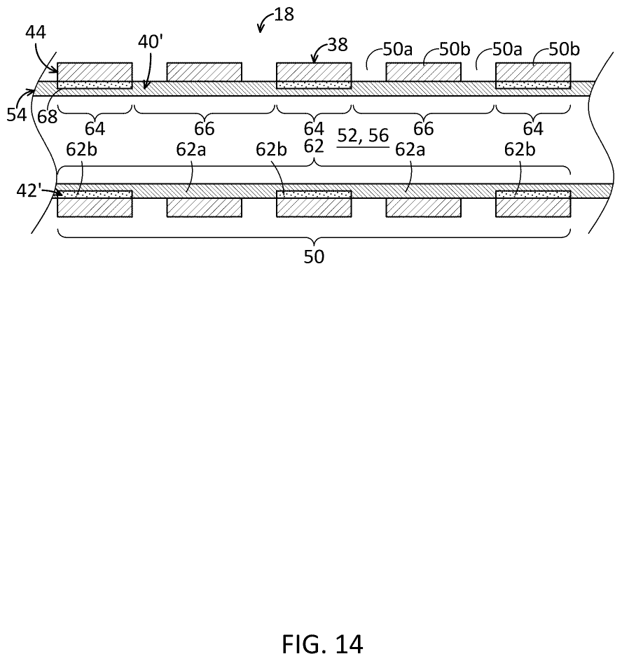

Although, in the embodiments illustrated in FIGS. 7 and 10-11, the tie layer 42 is disposed on an outer surface of the inner polymer liner 40, the tie layer may be embedded in the inner polymer liner. For example, in an alternative embodiment illustrated in FIG. 14, an inner polymer liner 40′ may have apertures 68 that entirely or partially extend through the polymer tube 54, and a tie layer 42′ may be embedded within the apertures 68. In this case, at least one discrete adhesion region 64 is formed between the hypotube structure 38 and the inner polymer liner 40, and at least one non-adhesion region 66 is formed between the hypotube structure 38 and the inner polymer liner 40.

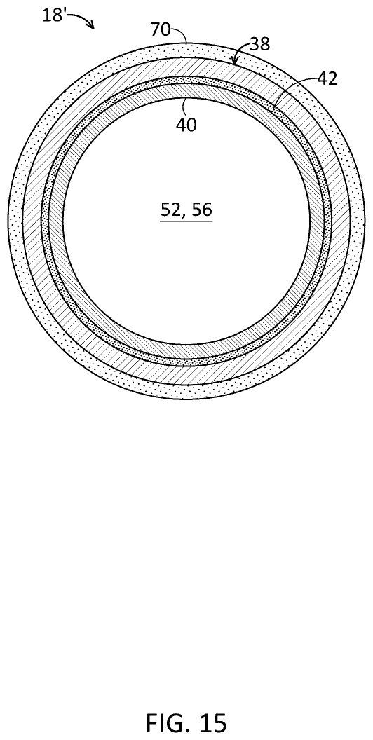

Although, in the embodiments illustrated in FIGS. 4-14, the catheter body 18 does not have a polymer jacket, an alternative embodiment of a catheter body 18′ illustrated in FIG. 15 comprises an outer polymer jacket 70 that is applied to the outer diameter of the hypotube structure 38 to provide a seal and to also minimize exterior surface roughness imparted by the hypotube pattern 50 of apertures 50a and solid elements 50b, while still providing flexibility. In some embodiments, the inner polymer liner 40 (or 40′) and tie layer 42 (or 42′) may be arranged in the manner illustrated in FIGS. 7, 10, 11, and 14 to form at least one discrete adhesion region 64 between the solid elements 62b of the hypotube structure 38 and the inner polymer liner 40 (or 40′).

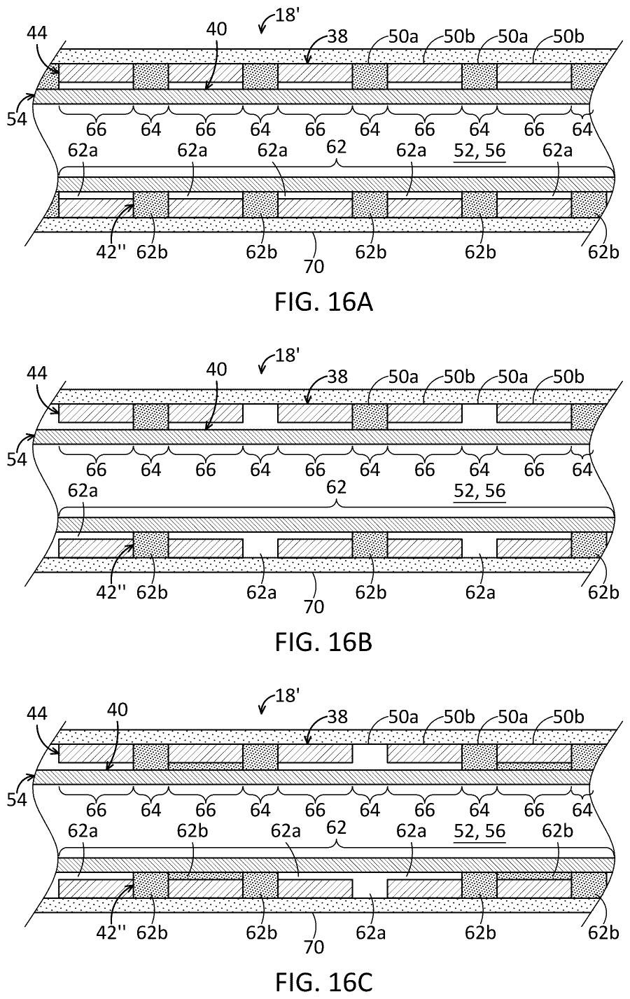

In other embodiments illustrated in FIG. 16A-16C, a tie layer 42″ has a tie layer pattern 62 of apertures 62a and solid elements 62b that is complementary to the hypotube pattern 50, such that the tie layer 42″ intermittently attaches the inner polymer liner 40 to the outer polymer jacket 70 through at least some of the apertures 50a of the hypotube pattern 50 of the hypotube structure 38. As a result, at least one discrete adhesion region 64 is formed between the hypotube structure 38 and the outer polymer jacket 70, and at least one non-adhesion region 66 is formed between the hypotube structure 38 and the inner polymer liner 40. In the illustrated embodiment, the tie layer pattern 62 is complementary to the hypotube pattern 50, such that several discrete adhesion regions 64 are formed between the hypotube structure 38 and the outer polymer jacket 70, and several non-adhesion regions 66 are formed between the hypotube structure 38 and the inner polymer liner 40. In the embodiment illustrated in FIG. 16A, the tie layer 42″ intermittently attaches the inner polymer liner 40 to the outer polymer jacket 70 through all of the apertures 50a of the hypotube pattern 50 of the hypotube structure 38, while in the embodiment illustrated in FIG. 16B, the tie layer 42″ intermittently attaches the inner polymer liner 40 to the outer polymer jacket 70 through all of the apertures 50a of the hypotube pattern 50 of the hypotube structure 38. In the embodiment illustrated in FIG. 16C, the tie layer 42″ also intermittently attaches the inner polymer liner 40 to the hypotube structure 38, such that discrete adhesion regions 64 are formed between some, but not all, of the solid elements 62b of the hypotube structure 38 and the inner polymer liner 40.

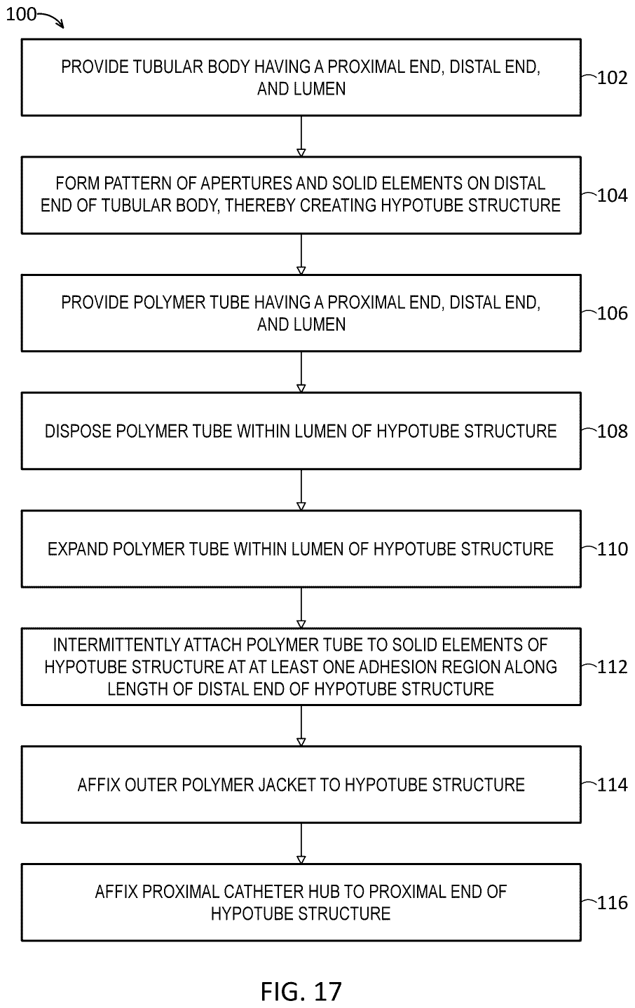

Having described the structure and arrangement of the intravascular catheter 10, one exemplary method 100 of manufacturing the intravascular catheter 10 will now be described with respect to FIG. 17.

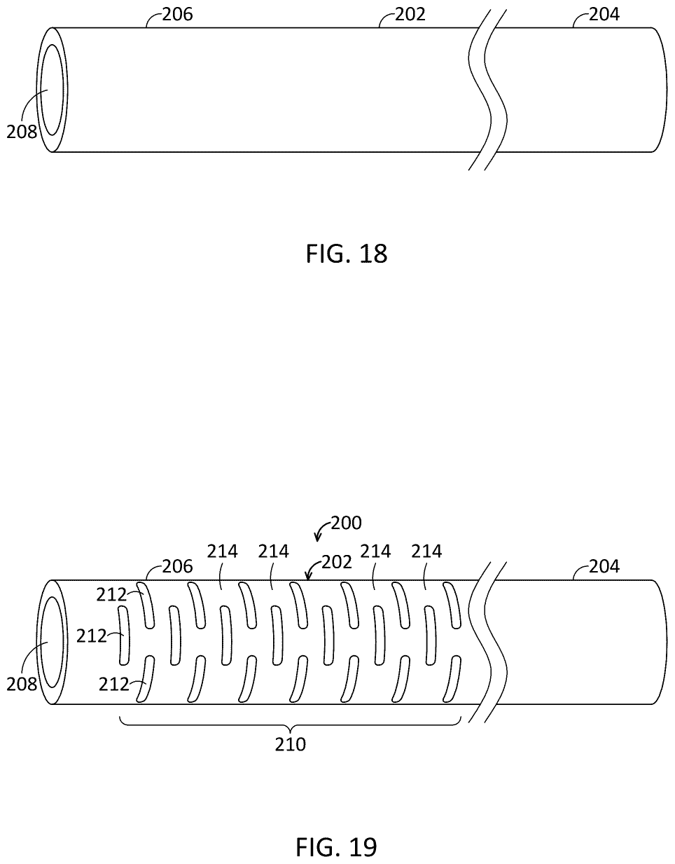

The method 100 comprises providing a hypotube structure. In particular, the method 100 comprises providing a tubular body 202 having a proximal end 204, a distal end 206, and a lumen 208 extending between the proximal end 204 and the distal end 206 (see FIG. 18) (step 102), and forming a pattern 210 of apertures 212 (e.g., slots) and solid elements 214 (e.g., struts) on the distal end 206 of the tubular body 202, e.g., by laser cutting, saw cutting (e.g., diamond grit embedded semiconductor dicing blade), etching, waterjet cutting, or electrical discharge machining, among other methods) (see FIG. 19) (step 104), thereby creating the hypotube structure 200.

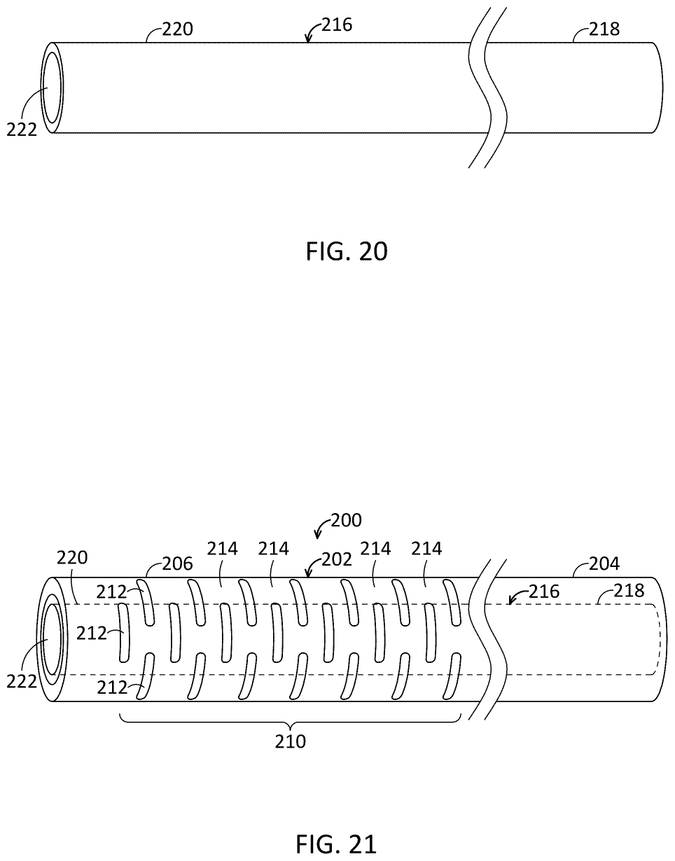

The method 100 further comprises providing a polymer tube 216 having a proximal end 218, a distal end 220, and a lumen 222 extending between the proximal end 218 and the distal end 220 (step 106) (see FIG. 20). The polymer tube 216 may be composed of, e.g., one or more of PTFE, ePTFE, fluoropolymer, PFA, FEP, and PE. The distal end 218 of the polymer tube 216 may have a wall thickness of 0.001″ or less. Preferably, the polymer tube 216 is unreinforced, such that the bending flexibility of the distal end of the resulting intravascular catheter is not degraded, and furthermore, such that the polymer tube 216 may be radially expanded. The method 100 may optionally comprise forming a pattern of apertures and solid elements on the distal end 218 of the polymer tube 216.

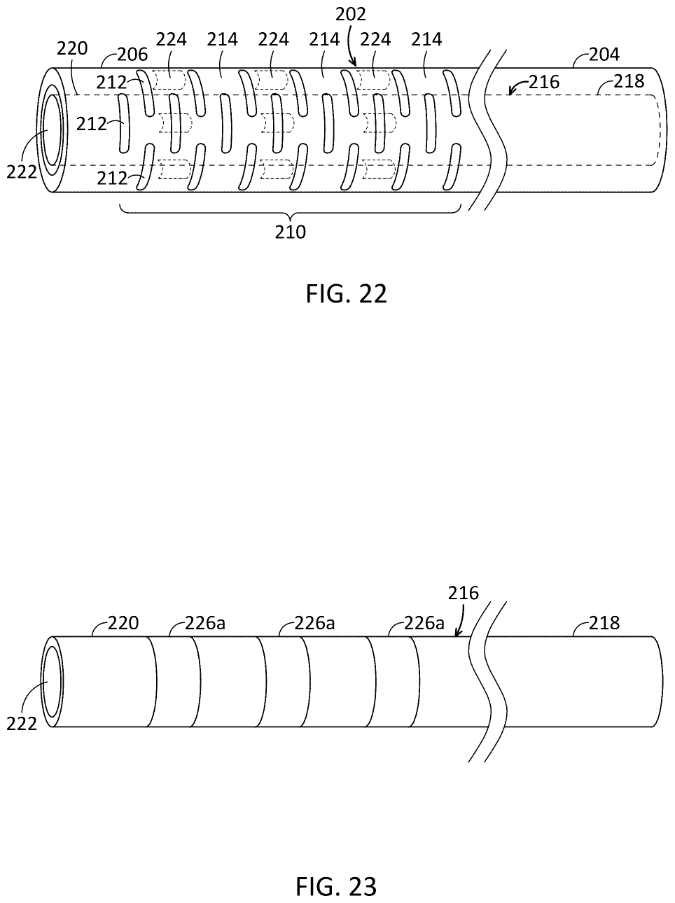

The method 100 further comprises disposing the polymer tube 216 within the lumen 206 of the hypotube structure 200 (see FIG. 21) (step 108). As a result, the polymer tube 216 serves as an inner polymer liner to the hypotube structure 200. The method further comprises radially expanding the polymer tube 216 within the lumen 206 of the hypotube structure 200, thereby decreasing the wall thickness of the polymer tube 216, as well as creating continuous intimate contact between the exterior of the polymer tube 216 and the interior of the hypotube structure 200 (step 110). The method 100 further comprises intermittently attaching the polymer tube 216 to the solid elements 214 of the hypotube structure 200 at at least one discrete adhesion region 224 along a length of the distal end 206 of the tubular body 202 (see FIG. 22) (step 112). Although in the illustrated method, the discrete adhesion region(s) 224 has a circumferential band pattern, it should be appreciated that the discrete adhesion region(s) 224 may alternatively take any suitable pattern, including a spiral pattern with a constant pitch/width or a variable pitch/width. Preferably, the total area of the discrete adhesion region(s) 224 is equal to or less than 75%, more preferably, equal to or less than 50%, and even more preferably, equal to or less than 25%, of the total area of the inner surface of the solid elements 214 of the hypotube structure 200 along the distal end 206 of the hypotube structure 200.

The polymer tube 216 may be intermittently attached to the solid elements 214 of the hypotube structure 200 using a tie layer 226, either during or after the expansion of the polymer tube 216 within the lumen 206 of the hypotube structure 200, which can be accomplished in any variety of manners.

In one method, the tie layer 226 is applied to the polymer tube 216 prior to disposing the polymer tube 216 within the lumen 206 of the hypotube structure 200, in which case, the polymer tube 216 may be intermittently attached to the solid elements 214 of the hypotube structure 200 via the tie layer 226 during expansion of the polymer tube 216 within the lumen 206 of the hypotube structure 200.

In one embodiment, a tie layer 226a may be applied as a positive pattern of adhesive material (i.e., an additive process) onto the outer surface of the polymer tube 216 to create a positive adhesive pattern (corresponding to the discrete adhesion region(s) 224), as illustrated in FIG. 23. The positive pattern of adhesive material may be composed of any of the aforementioned materials (e.g., polyurethane, Pebax®, or nylon) or polymers having a higher stiffness than typical tie-layer polymers, in addition to an adhesive, such as, e.g., heat-activated adhesives, catalyst-activated adhesives, solvent-activated adhesives, etc. The positive pattern of adhesive material may be applied to the outer surface of the polymer tube 216 by, e.g., applying a patterned mask (negative of the positive adhesive pattern) over the outer surface of the polymer tube 216, uniformly applying an adhesive material over the patterned mask (e.g., by dispersion coating, e.g., film casting or dip coating, or such as spraying), and then removing the patterned mask from the polymer tube 216, three-dimensional (3D) printing, ink-jet printing, etc. Although in the illustrated method, the tie layer 226a has a circumferential band pattern, it should be appreciated that the tie layer 226a may alternatively take any suitable pattern, including a spiral pattern with a constant pitch/width or a variable pitch/width.

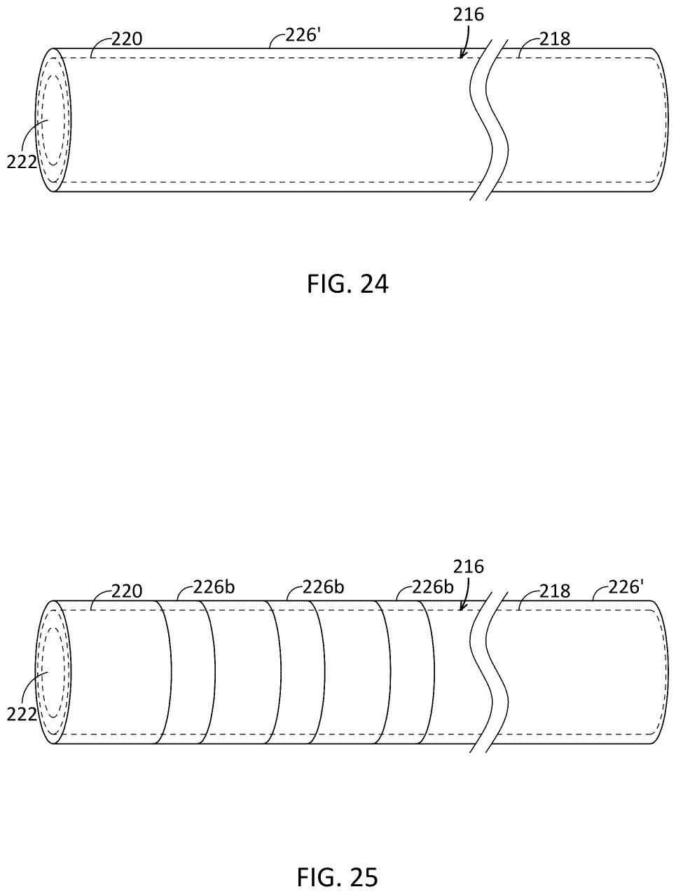

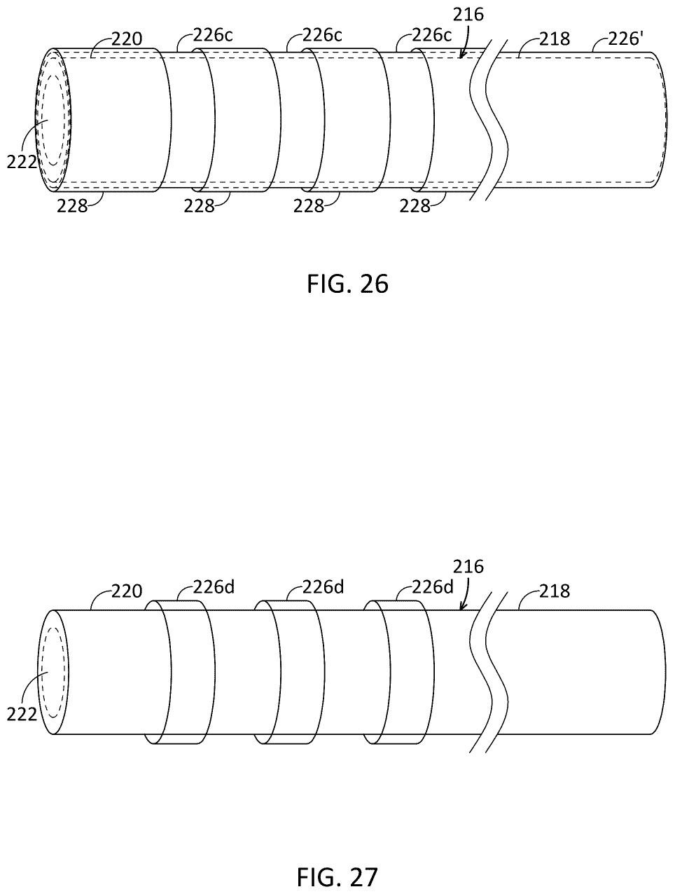

In other embodiments, a continuous tie layer 226′ may first be applied to the outer surface of the polymer tube 216 using a suitable process (e.g., by dispersion coating, e.g., film casting or dip coating, or such as spraying), as illustrated in FIG. 24, and then an adhesive pattern 224b (at least a portion of which that intersects with the solid elements 214 of the hypotube structure 200 corresponding to the discrete adhesion region(s) 224) may be formed on the continuous tie layer 226′ (e.g., by melting or otherwise activating adhesive properties of regions of the continuous tie layer 226′ corresponding to the adhesive pattern 224b), as illustrated in FIG. 25; or a positive pattern of a non-adhesive material (i.e., an additive process) 228 on the continuous tie layer 226′, thereby forming a pattern of adhesive material 226c (at least a portion of which that intersects with the solid elements 214 of the hypotube structure 200 corresponding to the discrete adhesion region(s) 224) outside of the positive pattern of non-adhesive material 228, as illustrated in FIG. 26; or ablating a negative pattern 230 (i.e., a subtractive process) within the continuous tie layer 226′, thereby forming a positive pattern of adhesive material 226d outside of the negative pattern 230 (at least a portion of which that intersects with the solid elements 214 of the hypotube structure 200 corresponding to the discrete adhesion region(s) 224), as illustrated in FIG. 27. Notably, photolithography can be used to create a pattern of active/inactive areas, or to condition a photosensitive material in a positive or negative pattern. Either the exposed or unexposed material is activated as an adhesive, or is de-activated, or is subsequently removed, with the remaining material acting as an adhesive.

In another method, the tie layer 226 is applied to the polymer tube 216 subsequent to disposing the polymer tube 216 within the lumen 206 of the hypotube structure 200, in which case, the polymer tube 216 may be intermittently attached to the solid elements 214 of the hypotube structure 200 via the tie layer 226 after expansion of the polymer tube 216 within the lumen 206 of the hypotube structure 200.

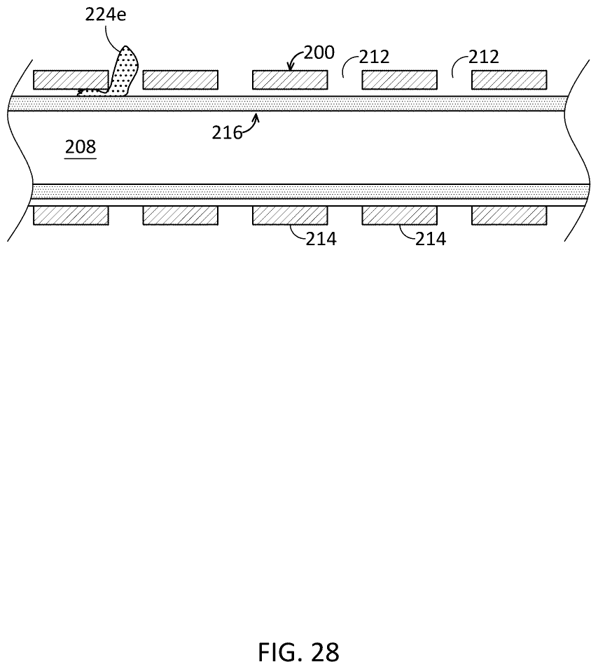

In one embodiment, a liquid adhesive is applied through the apertures 212 of the hypotube structure 200, such that the liquid adhesive seeps between the solid elements 214 of the hypotube structure 200 and the polymer tube 216, thereby creating an adhesive pattern 224e (at least a portion of which that intersects with the solid elements 214 of the hypotube structure 200 corresponding to the discrete adhesion region(s) 224), as illustrated in FIG. 28.

The method 100 optionally comprises affixing an outer polymer jacket (not shown) to the exterior of the hypotube structure 200 (step 114). In this case, the polymer tube 216 may be intermittently attached to the outer polymer jacket at at least one discrete adhesion region along a length of the distal end 206 of the tubular body 202. In this case, instead of intermittently attaching the polymer tube 216 to the solid elements 214 of the hypotube structure 200 via the tie layer 226 in step 112, the polymer tube 216 may be intermittently attached to the outer polymer jacket via the tie layer 226. In other embodiments, the polymer tube 216 may be intermittently attached to both the solid elements 214 of the hypotube structure 200 and the outer polymer jacket via the tie layer 226.

Lastly, the method 100 comprises affixing a proximal catheter hub 220 to the proximal end 204 of the hypotube structure 200 (not shown) (step 116).

Although particular embodiments have been shown and described herein, it will be understood by those skilled in the art that they are not intended to limit the disclosed inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

What is claimed is:

1. A method of manufacturing an intravascular catheter, comprising:
providing a hypotube structure having a tubular body with a proximal end, a distal end, a pattern of apertures and solid elements disposed on the distal end of the tubular body, and a hypotube lumen extending between the proximal end and the distal end of the tubular body;
providing a polymer tube having a tube lumen;
providing an outer polymer jacket;
affixing the outer polymer jacket to an exterior surface of the hypotube structure;
disposing the polymer tube within the hypotube lumen;
radially expanding the polymer tube within the hypotube lumen; and
intermittently attaching the polymer tube to at least some of the solid elements of the hypotube structure and/or the outer polymer jacket at at least one discrete adhesion region along a length of the distal end of the tubular body.

2. The method of claim 1, wherein the polymer tube is intermittently attached to the solid elements of the hypotube structure at the at least one discrete adhesion region along the length of the distal end of the tubular body.

3. The method of claim 2, wherein the radially expanded polymer tube is in intimate contact with the entirety of all of the solid elements of the hypotube structure.

4. The method of claim 2, wherein intermittently attaching the polymer tube to the solid elements of the hypotube structure comprises applying a liquid adhesive through the apertures of the hypotube structure, such that the liquid adhesive seeps between the solid elements of the hypotube structure and the polymer tube.

5. The method of claim 2, wherein intermittently attaching the polymer tube to the solid elements of the hypotube structure comprises applying a tie layer to the polymer tube prior to disposing the polymer tube within the hypotube lumen, such that radially expanding the polymer tube within the hypotube lumen intermittently attaches the polymer tube to the solid elements of the hypotube structure via the tie layer.

6. The method of claim 1, wherein the polymer tube is intermittently attached to the outer polymer jacket at the at least one discrete adhesion region along the length of the distal end of the tubular body.

7. The method of claim 6, wherein the polymer tube is further intermittently attached to the solid elements of the hypotube structure at the at least one discrete adhesion region along the length of the distal end of the tubular body.

8. The method of claim 6, wherein polymer tube is not attached to the solid elements of the hypotube structure.

9. The method of claim 6, wherein intermittently attaching the polymer tube to the outer polymer jacket comprises applying a tie layer to the polymer tube prior to disposing the polymer tube within the hypotube lumen, such that radially expanding the polymer tube within the hypotube lumen intermittently attaches the polymer tube to the outer polymer jacket through the apertures of the hypotube structure via the tie layer.

10. A method of manufacturing an intravascular catheter, comprising:
providing an elongate tubular body with a proximal end, a distal end, and a tubular body lumen extending between the proximal end and the distal end of the tubular body;
providing a polymer tube having a tube lumen;
disposing the polymer tube within the tubular body lumen;
radially expanding the polymer tube within the tubular body lumen; and
intermittently attaching the polymer tube to the tubular body at at least one discrete adhesion region along a length of the distal end of the tubular body.

11. The method of claim 10, wherein the polymer tube is unreinforced.

12. The method of claim 10, wherein a distal end of the radially expanded polymer tube has a wall thickness of 0.001″ or less.

13. The method of claim 10, wherein the radially expanded polymer tube is in continuous intimate contact with the tubular body.

14. The method of claim 10, further comprising applying a tie layer to the polymer tube, wherein the tie layer intermittently attaches the polymer tube to the tubular body at the at least one discrete adhesion region along the length of the distal end of the tubular body.

15. The method of claim 14, wherein the tie layer is applied to the polymer tube prior to disposing the polymer tube within the tubular body lumen.

16. The method of claim 15, wherein the tie layer is applied to an outer surface of the polymer tube.

17. The method of claim 16, wherein applying the tie layer to the outer surface of the polymer tube comprises dispersion coating the tie layer on the outer surface of the polymer tube.

18. The method of claim 16, wherein applying the tie layer to the outer surface of the polymer tube comprises applying a positive pattern of adhesive material on the outer surface of the polymer tube, wherein at least a portion of the positive pattern of adhesive material corresponds to the at least one discrete adhesion region.

19. The method of claim 16, wherein the tie layer is a continuous tie layer.

20. The method of claim 19, further comprising forming an adhesive pattern onto the continuous tie layer, wherein at least a portion of the adhesive pattern corresponds to the at least one discrete adhesion region.

21. The method of claim 20, wherein forming the adhesive pattern onto the continuous tie layer comprises melting or otherwise activating adhesive properties of regions of the continuous tie layer corresponding to the adhesive pattern.

22. The method of claim 20, further comprising applying a positive pattern of a non-adhesive material on the continuous tie layer, thereby forming a negative pattern of adhesive material outside of the positive pattern of non-adhesive material, wherein at least a portion of the positive pattern of adhesive material corresponds to the at least one discrete adhesion region.

23. The method of claim 19, further comprising ablating a negative pattern within the continuous tie layer, thereby forming a positive pattern of adhesive material outside of the negative pattern, wherein at least a portion of the positive pattern of adhesive material corresponds to the at least one discrete adhesion region.

24. The method of claim 15, further comprising ablating a negative pattern within the polymer tube to form a negative polymer tube pattern, wherein applying the tie layer to the polymer tube comprises disposing adhesive material in the negative polymer tube pattern, thereby forming a pattern of adhesive material, wherein at least a portion of the adhesive pattern corresponds to the at least one discrete adhesion region.

25. The method of claim 14, wherein the tie layer is applied to the polymer tube subsequent to disposing the polymer tube within the tubular body lumen.
